(19) United States
(12) Patent Application Publication
(10) Pub. No.: US 2015/0073468 A1
(43) Pub. Date: Mar. 12, 2015

(54) BALLOON FOR MEDICAL DEVICE

(71) Applicant: Covidien LP, Mansfield, MA (US)

(72) Inventor: Kumin Yang, Plymouth, MN (US)

(21) Appl. No.: 14/306,503

(22) Filed: Jun. 17, 2014

Related U.S. Application Data
(60) Provisional application No. 61/837,375, filed on Jun. 20, 2013.

(51) Int. Cl. A61L 31/04

(52) U.S. Cl. CPC A61L 31/041
USPC 606/194

(57) ABSTRACT

A transluminal balloon for a medical device may include a longitudinal structural portion including at least a first structural layer, the first structural layer including a polyether block amide co-polymer including a polyether, and a polyamide other than nylon 12. A transluminal balloon may include a longitudinal structural portion including an inner structural layer including a first polyether block amide co-polymer, an intermediate structural layer including a polyamide or copolyamide, and an outer structural layer a second polyether block amide co-polymer different from the first polyether block amide co-polymer. The inner and outer structural layers have hoop ratios greater than the intermediate structural layer, and the inner structural layer has a hoop ratio that is greater than or less than the outer structural layer.

### FIELD OF THE DISCLOSURE

The present invention generally relates to a balloon for a medical device and a medical device including a balloon.

### BACKGROUND OF THE DISCLOSURE

Expandable medical balloons are employed in a variety of medical procedures, including balloon angioplasty, as well as for delivery of medical devices to the treatment site, such as stent delivery.

### SUMMARY OF THE DISCLOSURE

In one aspect, a transluminal balloon for a medical device may generally comprise a longitudinal structural portion including at least a first structural layer, the first structural layer comprising a polyether block amide co-polymer comprising a polyether, and a polyamide other than nylon 12. In another aspect, a transluminal balloon may generally comprise a longitudinal structural portion comprising an inner structural layer comprising a first polyether block amide co-polymer, an intermediate structural layer comprising a polyamide or copolyamide, and an outer structural layer a second polyether block amide co-polymer different from the first polyether block amide co-polymer. The inner and outer structural layers have hoop ratios greater than the intermediate structural layer, and the inner structural layer has a hoop ratio that is greater than or less than the outer structural layer. In yet another aspect, a transluminal balloon for a medical device may generally comprise a longitudinal structural portion comprising an inner structural layer comprising a first polyester block copolymer thermoplastic elastomer comprising a hard segment of polyester and a soft segment of polyether, an intermediate structural layer comprising a polyester or copolyester, and an outer structural layer comprising a second polyester thermoplastic block copolymer comprising a hard segment of polyester and a soft segment of polyether. The first polyester block copolymer thermoplastic elastomer has a lower Shore D hardness than a Shore D hardness of the second polyester block copolymer thermoplastic elastomer polyester.

### BRIEF DESCRIPTION OF THE DRAWINGS

FIG. 1 is an elevation of an embodiment of a bi-layer with parts broken away to show internal construction;

FIG. 2 is a cross section of the balloon in FIG. 1 taken along the line 2-2 in FIG. 1, a guidewire lumen being omitted for purposes of illustration;

FIG. 3 is longitudinal section of an embodiment of a tri-layer balloon;

FIG. 4 is a cross section of the balloon in FIG. 3 taken along the line 4-4 in FIG. 3; and

FIG. 5 is a longitudinal section of an embodiment of a monolayer balloon.

Corresponding reference characters indicate corresponding parts throughout the drawings.

### DETAILED DESCRIPTION OF EMBODIMENTS

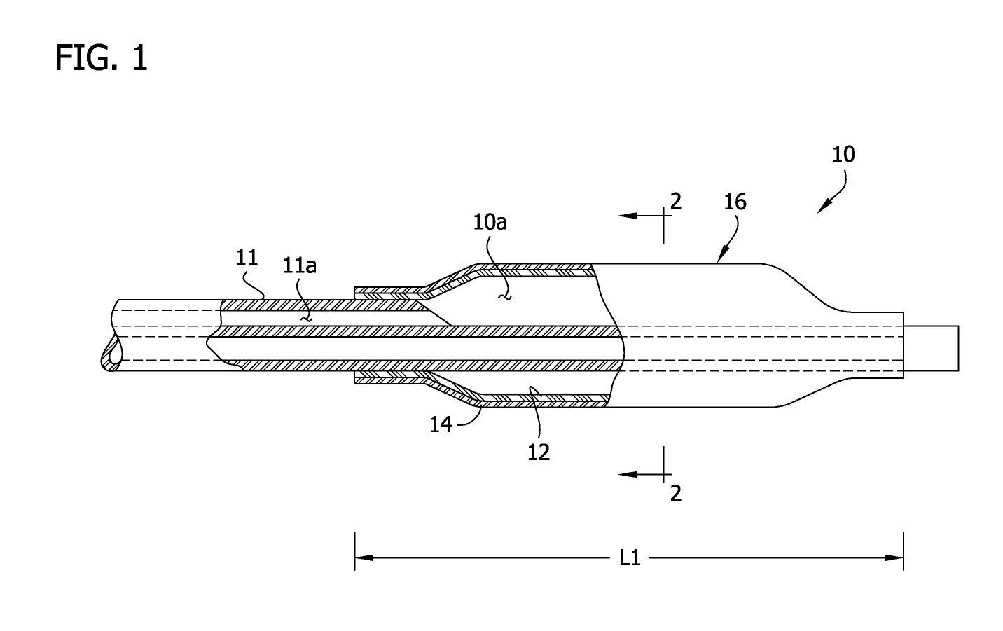

The present disclosure is directed to a transluminal balloon for a medical device. In one embodiment, the balloon is generally elongate and includes a generally tubular balloon body and cone segments at opposite longitudinal ends of the balloon body. The balloon defines an interior chamber for receiving fluid therein to expand an outer circumference of the balloon body. With respect to any or all of the below-described embodiments of the present disclosure, the transluminal balloon may be secured to a catheter (as shown in FIG. 1) or other medical device and configured for introduction along and inflation (or circumferential expansion) within a blood vessel for treating vascular stenosis. For example, the transluminal balloon may be configured for introduction along and inflation within one or more of peripheral arteries and veins, coronary arteries and veins, renal arteries and veins, cerebral arteries and veins, and carotid artery. In other examples, the transluminal balloon may be configured for introduction along and inflation within other body lumens for treating stenosis of those lumens.

With respect to any or all of the below-described embodiments of the present disclosure, a longitudinal structural portion of the transluminal balloon includes one or more structural layers of polymeric material. As used herein, the longitudinal structural portion may be, for example, a longitudinal segment of the balloon that encompasses less than an entire length of the balloon and the longitudinal segment may be continuous or non-continuous along the length of the balloon, or in another example, the longitudinal structural portion may encompass the entire length of the balloon. A structural layer is a circumferential layer of polymeric material that gives the balloon its structure and mechanical properties. Each structural layer is a thermoplastic or thermoplastic elastomer polymer, which, in one example, may be formed by extrusion or co-extrusion and subsequent blow molding to form the balloon. In at least some examples of the below-described embodiments, the balloon may be free from fibers, carbon nanotubes, clay platelets, and other reinforcing materials, although, unless stated otherwise, it is contemplated that the structural layers may include such reinforcing materials. The balloon may include other “layers,” which are not considered “structural layers.” For example, a lubricant coating or layer may be applied to the outermost structural layer of the longitudinal structural portion of the balloon (i.e., the exterior of the balloon), however, the lubricant coating is not considered a structural layer if its sole function is to provide lubricity to the balloon. Moreover, the balloon may include a non-structural layer defining the interior surface of the balloon that is not a structural layer and/or the balloon may include one or more non-structural layers that are radially between (i.e., sandwiched between) adjacent structural layers.

In one non-limiting embodiment of the present disclosure, a longitudinal structural portion of the balloon (e.g., the balloon body or the entire balloon length) comprises at least one structural layer including a polyether block amide (PEBA) co-polymer having polyamide segment other than nylon 12 that is made from the oligomerization of a monomer other than laurolactam (hereinafter referred to as “X-PEBA”). For example, the polyamide segment may be a polyamide that is mechanically stronger than nylon 12 and/or produces a PEBA that is mechanically stronger than a PEBA including nylon 12. In at least one example, the polyamide segment of X-PEBA may include nylon 13; nylon 6, 13; nylon 10, 13; nylon 11; nylon 6, 11; or nylon 10, 11. The X-PEBA may have a Shore D hardness of greater than 72D, such as about 74D, or from about 72D to about 75D. Such a balloon having at least one structural layer including X-PEBA is referred to hereinafter as “X-PEBA balloon.” In one non-limiting example, a longitudinal structural portion of the X-PEBA balloon (e.g., at least the balloon body or the entire balloon length) comprises a single structural layer (i.e., one and only one structural layer) including X-PEBA (hereinafter referred to as “monolayer X-PEBA balloon”). As explained in more detail below, the single structural layer may consist essentially of X-PEBA or a blend thereof.

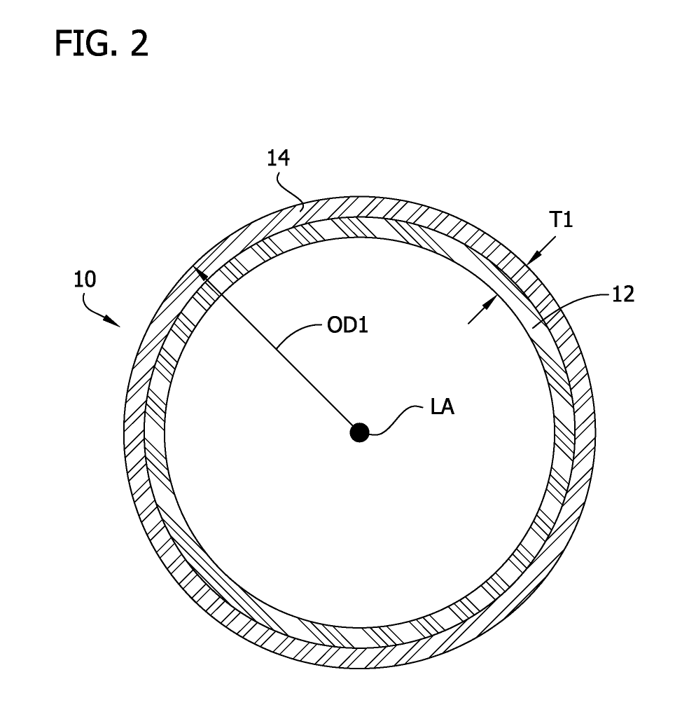

In another non-limiting example, at least a longitudinal structural portion of the X-PEBA balloon (e.g., the balloon body or the entire balloon) comprises two or more structural layers, wherein at least one of the structural layers includes X-PEBA (hereinafter referred to as “multi-layer X-PEBA balloon”). As explained in more detail below, the at least one structural layer of X-PEBA may consist essentially of X-PEBA or a blend thereof. At least one of the structural layers of the multi-layer X-PEBA balloon is an “inner structural layer,” and at least one of another structural layer is an “outer structural layer” relative to the specific inner structural layer. For all multi-structural layer balloon embodiments disclosed herein, the terms “inner structural layer” and “outer structural layer” are relative terms denoting the relative radial locations of respective structural layers with respect to a longitudinal axis (e.g., longitudinal axis A1 in FIG. 2) of the balloon. To clarify, a first structural layer of the longitudinal structural portion of the balloon is considered an “inner structural layer” with respect to a second structural layer of the longitudinal structural portion of the balloon if the first structural layer is radially inward of the second structural layer. Likewise, if the first structural layer is an inner structural layer with respect to the second structural layer, it is necessarily true that the second structural layer is an outer structural layer with respect to the first structural layer. It is not necessarily true, however, that inner and outer structural layers are the respective innermost and outermost structural layers of the balloon. In addition, it is not necessarily true that inner and outer structural layers are radially adjacent one another without an intermediate structural layer disposed between the inner and outer structural layers. That is, unless otherwise specified, the balloon may include one or more intermediate structural layers between structural layers that are considered inner and outer structural layers relative to one another.

In another non-limiting embodiment of the present disclosure, a longitudinal structural portion of a transluminal balloon for a medical device comprises at least one structural layer including a transparent amorphous polyamide based on aliphatic and/or cycloaliphatic blocks. Such a balloon is referred to hereinafter as a “transparent amorphous polyamide balloon.” In one non-limiting example, a longitudinal structural portion of a transparent amorphous polyamide balloon comprises a single structural layer, i.e., one and only one structural layer, including transparent amorphous polyamide (hereinafter referred to as a “monolayer amorphous polyamide balloon”). As explained in more detail below, the monolayer of amorphous polyamide may consist essentially of a transparent amorphous polyamide or a blend thereof. In another non-limiting example, a transparent amorphous polyamide balloon comprises two or more structural layers, at least one of the structural layers including transparent amorphous polyamide (hereinafter referred to as a “multi-layer transparent amorphous polyamide balloon”). As explained in more detail below, the at least one structural layer of transparent amorphous polyamide may consist essentially of transparent amorphous polyamide or a blend thereof. One of the structural layers of the multi-structural layer amorphous polyamide balloon is an “inner structural layer,” and another of the structural layers is an “outer structural layer,” the meanings of which are provided above. As explained below, in one example a multi-structural layer balloon may comprise a structural layer including X-PEBA and another structural layer including transparent amorphous polyamide.

In another non-limiting embodiment of the present disclosure, a longitudinal structural portion of a transluminal balloon for a medical device comprises at least two structural layers, wherein at least one of the structural layers includes a copolyester and at least another structural layer includes polyester thermoplastic elastomer (polyester TPE) (hereinafter referred to as a “multi-layer polyester TPE balloon”). The at least one structural layer of copolyester may consist essentially of copolyester or a blend thereof. The at least one structural layer of polyester TPE may consist essentially of polyester TPE or a blend thereof. One of the structural layers of the multi-structural layer polyester TPE balloon is an “inner structural layer,” and another of the structural layers is an “outer structural layer,” the meanings of which are provided above.

In another non-limiting embodiment of the present disclosure, a longitudinal structural portion of a transluminal balloon for a medical device comprises an inner structural layer and an outer structural layer. The inner structural layer includes a first polyester thermoplastic elastomer, and the outer structural layer includes a second polyester thermoplastic elastomer having at least one property that is different than the first polyester thermoplastic elastomer. In one example, the first polyester thermoplastic elastomer has a Shore D hardness (e.g., Shore 72D) less than a Shore D hardness (e.g., Shore 82D) of the second polyester thermoplastic elastomer.

In another non-limiting embodiment of the present disclosure, a longitudinal structural portion of a transluminal balloon for a medical device comprising a single structural layer, i.e., one and only one structural layer (hereinafter referred to as a “monolayer balloon”). As set forth above, the single structural layer of the monolayer balloon may include X-PEBA. For example, the single structural layer may consist essentially of X-PEBA or a blend thereof. In other non-limiting examples, the single structural layer of the monolayer balloon may include transparent amorphous polyamide. For example, the single structural layer may consist essentially of transparent amorphous polyamide or a blend thereof.

Referring now to FIGS. 1 and 2, an illustrated embodiment of a multi-layer balloon for a medical device is generally indicated at 10. As shown in FIG. 1, this balloon 10 is secured to an elongate catheter body 11 adjacent a distal end thereof, thereby forming a balloon catheter for treating stenosis of a body lumen. The balloon 10 is generally elongate and includes an elongate, generally tubular balloon body, generally indicated at 16, and cone segments, generally indicated at 18, at opposite longitudinal ends of the balloon body. The balloon 10 defines an interior chamber or lumen 20 that is in fluid communication with an inflation lumen 11a defined by the catheter body 11. Fluid (such as saline) is introduced into the catheter body lumen 11a adjacent a proximal end (not shown) of the catheter body 11 for delivering the fluid into the interior chamber 20 of the balloon to thereby inflate or expand the balloon to a desired internal pressure. The use of the balloon catheter for treating stenosis of a body lumen is generally understood by one having ordinary skill in the art.

The multi-structural layer balloon illustrated in FIGS. 1 and 2 is more specifically a “bi-layer balloon” because it includes a longitudinal structural portion comprising two polymeric structural layers: an inner structural layer 12 and an outer structural layer 14. In the illustrated embodiment, the longitudinal structural portion encompasses the entire length L1 of the balloon, although in other embodiments, the longitudinal structural portion may encompass less than the entire length L1 of the balloon, such as, but not limited to, the balloon body 16 and not the cone segments 18. Moreover, because this is a bi-layer balloon (i.e., it has two and only two structural layers), the inner structural layer 12 constitutes a radially innermost structural layer, and the outer structural layer 14 constitutes a radially outermost structural layer. In a first example, the inner structural layer 12 includes X-PEBA, and the outer structural layer 14 includes polyamide or copolyamide. The inner and outer structural layers 12, 14 may include other polymers, such as, but not limited to, polymers described below herein.

With respect to the first example, the inner structural layer 12 includes X-PEBA. The X-PEBA includes a polyamide segment (i.e., a hard segment) and a polyether segment (i.e., a soft segment), such as poly(tetramethylene oxide) (PTMO). The PA segment may be a polyamide other than nylon 12 that is made from the oligomerization of a monomer other than laurolactam. In particular, the polyamide segment may be a polyamide that is mechanically stronger than nylon 12 and/or produces a PEBA that is mechanically stronger than a PEBA including nylon 12. In at least one example, the polyamide segment of X-PEBA may include nylon 13; nylon 6, 13; nylon 10, 13; nylon 11; nylon 6, 11; or nylon 10, 11. The X-PEBA may have a Shore D hardness of greater than 72D, such as about 74D, or from about greater than 72D to about 75D. A suitable X-PEBA is available from Arkema, Inc. of Colombes, France. The inner structural layer 12 may consist essentially of X-PEBA or a blend thereof. With respect to the X-PEBA blend, X-PEBA may be blended with polyamide. Exemplary polyamides for use in the X-PEBA blend include, but are not limited to, aliphatic polyamides, such as nylon 12; nylon 11; nylon 9; nylon 6; nylon 6, 12; nylon 6, 11; nylon 6, 9; nylon 6,6; nylon 10, 10; nylon 10, 12; nylon 6, 10; nylon 13; nylon 6, 13; nylon 10, 13; nylon 11; nylon 6, 11; or nylon 10, 11, or aromatic polyamides. Additional exemplary polyamides include transparent amorphous polyamides having a segment such as an aliphatic segment, an aromatic segment, or a cycloaliphatic segment. The selected transparent amorphous polyamide may have a Shore D durometer value greater than 80D, and a glass transition temperature greater than 100° C. or greater than 120° C. A suitable, non-limiting example of transparent amorphous polyamide includes GRILAMID® TR55 LX2, commercially available from EMS-GRIVORY of Switzerland. The properties for GRILAMID® TR55 LX, commercially available from EMS-GRIVORY of Switzerland, are provided in Table 1 (below).

TABLE 1

| | | | | | | Grilamid TR |
|---|---|---|---|---|---|---|
| PROPERTIES | | | Norm | Unit | State | 55 LX |
| Mechanical Properties | | | | | | |
| Tensile E-Modulus | 1 | mm/min | ISO527 | MPa | cond. | 1900 |
| Tensile strength at yield | 50 | mm/min | ISO527 | MPa | cond. | 70 |
| Elongatoin at yield | 50 | mm/min | ISO527 | % | cond. | 6 |
| Tensile strength at break | 50 | mm/min | ISO527 | MPa | cond. | 40 |
| Elongatoin at break | 50 | mm/min | ISO527 | % | cond. | >50 |
| Impact strength | Charpy, 23° c. | | ISO 179/2-1eU | KJ/m² | cond. | No break |
| Impact strength | Charpy, −30° c. | | ISO 179/2-1eU | KJ/m² | cond. | No break |
| Notched impact strength | Charpy, 23° c. | | ISO 179/2-1eA | KJ/m² | cond. | 9 |
| Notched impact strength | Charpy, −30° c. | | ISO 179/2-1eA | KJ/m² | cond. | 8 |
| Ball indentatoin hardness | | | ISO 2039-1 | MPa | cond. | 110 |
| Thermal Properties | | | | | | |
| Glass transition | DSC | | ISO 11357 | ° C. | dry | 110 |
| temperatures | | | | | | |
| Heat deflection | 1.80 | MPa | IS0 75 | ° C. | dry | 80 |
| temperature HOT/A | | | | | | |
| Heat deflection | 0.45 | MPa | IS0 75 | ° C. | dry | 90 |
| temperature HDT/C | | | | | | |
| Thermal expansion | 23-55° | C. | ISO 11359 | 10⁴/k | dry | 0.9 |
| coefficient long | | | | | | |
| Thermal expansion | 23-55° | C. | ISO 11359 | 10⁴/k | dry | 0.9 |
| coefficient trans. | | | | | | |
| Maximum usage | long term | | IS0 2578 | ° C. | dry | 80 |
| temperature | | | | | | |
| Maximum usage | short term | | IS0 2578 | ° C. | dry | 95 |
| temperature | | | | | | |
| Electrical Properties | | | | | | |
| Dielectric strength | | | IEC 60243-1 | kV/mm | cond. | 32 |
| Comparative tracking | CTI | | IEC 60112 | — | cond. | 600 |
| index | | | | | | |
| Specific volume | | | IEC60093 | Ω m | cond. | 10¹¹ |
| resistivity | | | | | | |
| Specific surface | | | IEC60093 | Ω | cond. | 10¹² |
| resistivity | | | | | | |
| General Properties | | | | | | |
| Density | | | ISO 1183 | g/cm³ | dry | 1.04 |
| Flammability (UL94) | 0.8 | mm | ISO 1210 | rating | — | HB |
| Water absorptoin | 23° | C./sat. | ISO62 | % | — | 2.5 |

| | | | | | |
|---|---|---|---|---|---|
| Moisture absorption | 23° C./50% r.h. | ISO62 | % | — | 1 |
| Linear mould shrinkage | long. | ISO294 | % | dry | 0.50 |
| Linear mould shrinkage | trans. | ISO294 | % | dry | 0.60 |

Product-nomenclature acc. ISO 1874: PA 12/MACMI + PA 12, GHLT, 14-020

GRILAMID® TR55 LX2 may have similar properties as GRILAMID® TR55 LX, although GRILAMID® TR55LX2 has two times the amount of nylon 12 than GRILAMID® TR55 LX, which may be beneficial when forming the balloon. It is expected that GRILAMID® TR55 LX2 will have greater elongation values and lower tensile values than GRILAMID® TR55LX. The X-PEBA blend may include other types of polyamides, such as nylon 12 or a material including nylon 12, and/or may include other types of polymers. In one example, the X-PEBA blend may be more than 50% by weight X-PEBA, or more than or equal to about 60% by weight X-PEBA, or more than or equal to about 70% by weight X-PEBA, or more than or equal to about 80% by weight X-PEBA, or more than or equal to about 85% by weight X-PEBA, or more than or equal to 90% by weight X-PEBA, or more than or equal to 95% by weight X-PEBA.

With respect to the outer structural layer 14 of the illustrated embodiment, the outer structural layer 14 may consist essentially of polyamide or may consist essentially of polyamide blend. In one example, the polyamide of the outer structural layer may be an aliphatic polyamide, such as nylon 12; nylon 11; nylon 9; nylon 6; nylon 6, 12; nylon 6, 11; nylon 6, 9; nylon 6,6; nylon 10, 10; nylon 10, 12; nylon 6, 10; nylon 13; nylon 6, 13; nylon 10, 13; nylon 11; nylon 6, 11; or nylon 10, 11, or aromatic polyamides. In another example, the polyamide of the outer structural layer 14 may be transparent amorphous polyamide, such as the example of transparent amorphous polyamide disclosed above with respect to the X-PEBA blend. The outer structural layer 14 may include other types of polyamides and/or may include other types of polymers.

In one example of the bi-layer X-PEBA balloon 10, the body 16 of the balloon may have a maximum outer diameter OD1 (upon inflation or expansion) from about 3.0 mm to about 12.0 mm. For example, the balloon body 16 OD1 may be about 3.0 mm, or about 3.5 mm, or about 4.0 mm, or about 4.5 mm, or about 5.0 mm, or about 5.5 mm, or about 6.0 mm, or about 6.5 mm, or about 7.0 mm, or about 7.5 mm, or about 8.0 mm, or about 8.5 mm, or about 9.0 mm, or about 9.5 mm, or about 10.0 mm, or about 10.5 mm, or about 11.0 mm, or about 11.5 mm, or about 12.0 mm, or about 12.5 mm, among other diameters. The body 16 of the bi-layer X-PEBA balloon 10 may have a working length WL1 (FIG. 1) from about 15 mm to about 200 mm. The body 16 of the X-PEBA balloon 10 has a total wall thickness T1 (FIG. 2) of about 0.025 inch (0.635 mm). In other examples, the total wall thickness T1 of the body 16 may be from about 0.015 inch (0.381 mm) to about 0.040 inch (1.02 mm), or from about 0.020 inch (0.508 mm) to about 0.030 inch (0.762 mm), or from about 0.020 inch (0.508 mm) to about 0.025 inch (0.635 mm). A thickness of the outer structural layer 14 may be about 80% of a total wall thickness T1 of the body 16, and a thickness of the inner structural layer 12 may be about 20% of the total wall thickness of the body. In other examples, the thickness of the outer structural layer 14 may be from about 50% to about 90%, or from about 60% to about 85%, or from about 70% to about 80% of the total wall thickness T1 of the body 16, with the remaining percentage being the percentage of the wall thickness of the inner structural layer 12.

In one exemplary method, the bi-layer X-PEBA balloon 10 may be blow-molded from a preform or parison using blow molding techniques and processes or by other methods. The parison may be formed by co-extruding the inner and outer structural layers 12, 14 using coextrusion techniques and processes or by other methods. In at least one example, the longitudinal structural portion including the one or more structural layers is free from a tie or adhesive layer between the two structural layers 12, 14.

Referring still to FIGS. 1 and 2, in another example of the bi-layer balloon 10, the outer structural layer 14 of the of the longitudinal structural portion of the balloon includes copolyester and the inner structural layer includes polyester thermoplastic elastomer (“polyester TPE”), or the outer structural layer includes a first polyester TPE having a Shore D hardness and the inner structural layer includes a second polyester TPE having a Shore D hardness less than the Shore D hardness of the first polyester TPE.

With respect to the inner structural layer 12 of this bi-layer polyester TPE balloon example, the polyester TPE may be a block copolymer comprising a hard segment of polyester (e.g., polybutylene terephthalate (PBT), among other polyesters) and a soft segment of polyether. A suitable polyester TPE is HYTREL®, which is commercially available from E. I. du Pont de Nemours and Company, Wilmington, Del. The polyester TPE may have a Shore D hardness from about 30D to about 82D. The inner structural layer 12 may consist essentially of polyester TPE (e.g., HYTREL®) or a blend thereof. With respect to the polyester TPE blend, HYTREL® or another polyester thermoplastic elastomer may be blended with polyester and/or copolyester. Exemplary polyesters and copolyesters for use in the polyester TPE blend include, but are not limited to, polyethylene terephthalate (PET) and copolyesters thereof, and polybutylene terephthalate (PBT) and copolyesters thereof. For example, a suitable copolyester includes Array® EBM polyethylene terepthalate, commercially available from DAK Americas LLC, Chadds Ford, Pa. In one example, the polyester TPE blend may be more than 50% by weight polyester TPE, or more than or equal to about 60% by weight polyester TPE, or more than or equal to about 70% by weight polyester TPE, or more than or equal to about 80% by weight polyester TPE, or more than or equal to about 85% by weight polyester TPE, or more than or equal to 90% by weight polyester TPE, or more than or equal to 95% by weight polyester TPE.

With respect to the outer structural layer 14 of this bi-layer polyester TPE balloon example, the copolyester may be ARRAY 9921M copolyester, commercially available from DAK Americas. The outer structural layer 14 may consist essentially of the copolyester (e.g., ARRAY 9921M copolyester) or a blend thereof. In the example where the outer structural layer 14 includes the first polyester TPE, the first polyester TPE may be HYTREL®, as disclosed above, having a Shore D hardness greater than the Shore D hardness of the second polyester TPE of the inner structural layer 12, which may also be HYTREL®. For example, the first polyester TPE may be HYTREL® 82D and the second polyester TPE may be HYTREL® 72D. The outer structural layer 14 may include other types of copolyesters or other types of polymers.

In one example of the bi-layer polyester TPE balloon 10, the body 16 of the balloon may have a maximum outer diameter OD1 (upon inflation or expansion) from about 3.0 mm to about 12.0 mm. For example, the balloon body 16 OD1 may be about 3.0 mm, or about 3.5 mm, or about 4.0 mm, or about 4.5 mm, or about 5.0 mm, or about 5.5 mm, or about 6.0 mm, or about 6.5 mm, or about 7.0 mm, or about 7.5 mm, or about 8.0 mm, or about 8.5 mm, or about 9.0 mm, or about 9.5 mm, or about 10.0 mm, or about 10.5 mm, or about 11.0 mm, or about 11.5 mm, or about 12.0 mm, or about 12.5 mm, among other diameters. The body 16 of the bi-layer polyester TPE balloon 10 may have a working length WL1 (FIG. 1) from about 15 mm to about 200 mm. The body 16 of the polyester TPE balloon 10 has a total wall thickness T1 (FIG. 2) of about 0.020 inch (0.508 mm). In other examples, the total wall thickness T1 of the body 116 may be from about 0.015 inch (0.381 mm) to about 0.040 inch (1.02 mm), or from about 0.020 inch (0.508 mm) to about 0.030 inch (0.762 mm), or from about 0.020 inch (0.508 mm) to about 0.025 inch (0.635 mm). A thickness of the outer structural layer 14 may be about 80% of a total wall thickness T1 of the body 16, and a thickness of the inner structural layer 12 may be about 20% of the total wall thickness of the body. In other examples, the thickness of the outer structural layer 14 may be from about 50% to about 90%, or from about 60% to about 85%, or from about 70% to about 80% of the total wall thickness T1 of the body 16, with the remaining percentage being the percentage of the wall thickness of the inner structural layer 12.

In one exemplary method, the bi-layer layer polyester TPE balloon 10 may be blow-molded from a preform or parison using conventional blow molding techniques and processes or by other methods. The parison may be formed by co-extruding the inner and outer structural layers 12, 14 using conventional coextrusion techniques and processes or by other methods. In at least one example, the longitudinal structural portion of the body including the structural layers is free from a tie or adhesive layer between the two structural layers 12, 14.

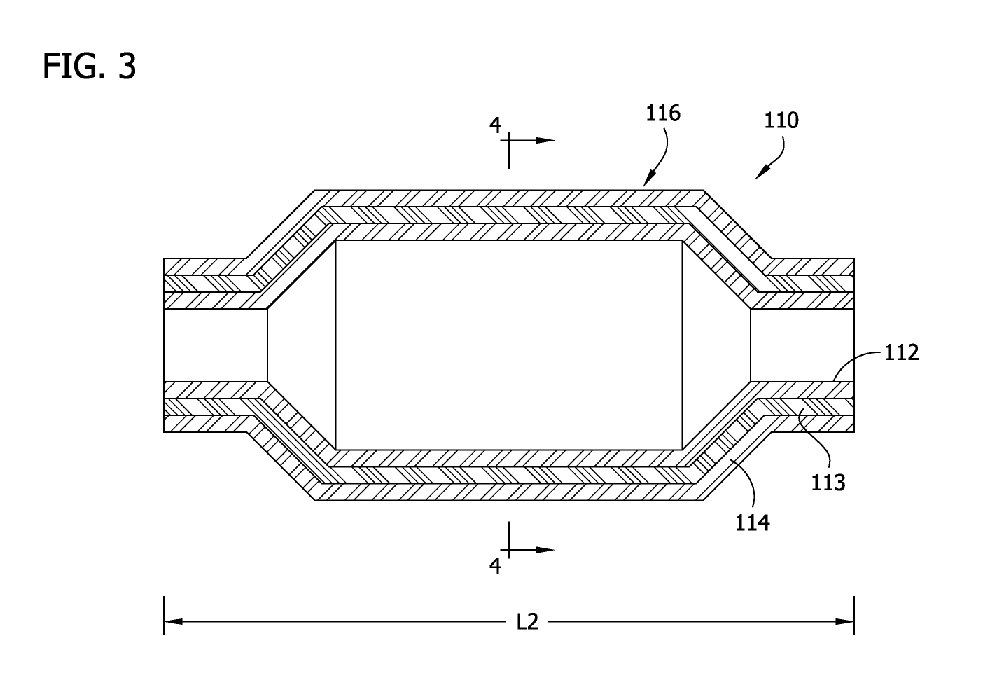

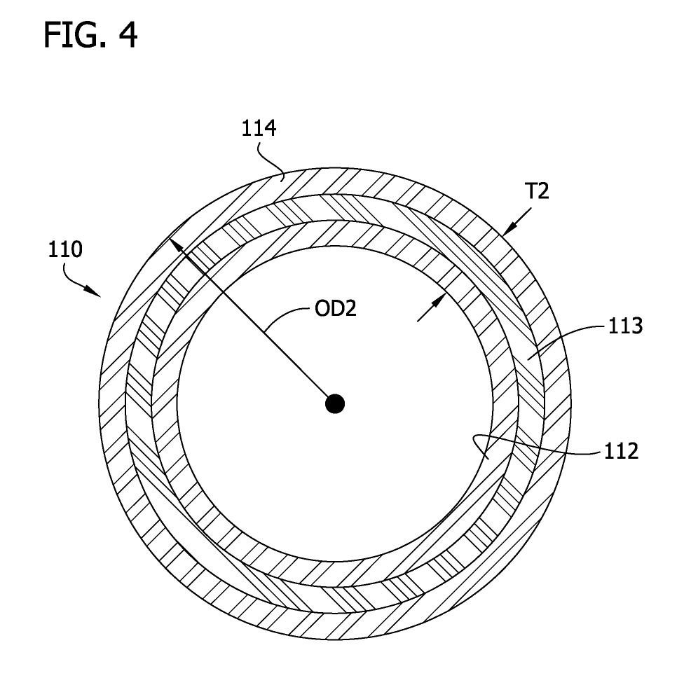

Referring to FIGS. 3 and 4, an illustrated embodiment of a multi-structural layer balloon for a medical device (e.g., a catheter) is generally indicated at 110. The multi-structural layer balloon illustrated in FIGS. 3 and 4 is more specifically a “tri-layer balloon” because it comprises a body 116 comprising three, and only three, polymeric structural layers: an innermost structural layer 112, an intermediate structural layer 113, and an outermost structural layer 114. Because this example is a tri-layer balloon, the innermost structural layer 112 is considered an inner structural layer relative to the intermediate and outermost structural layers 113, 114; the intermediate structural layer is considered an outer structural layer relative to the innermost structural layer 112; and the outermost structural layer 114 is considered an outer structural layer relative to the innermost and intermediate structural layers 112, 113.

In one example of the tri-layer balloon 110, the respective structural layers 112, 113, 114 have different hoop ratios. As used herein, the “hoop ratio” is defined as ratio of the balloon mold inner diameter over the extrusion tubing inner diameter. There exists a range of hoop ratio values for a given polymer where balloons can be blow-molded for optimum mechanical properties (radial expansion) and manufacturability. For example, the innermost and outermost structural layers 112, 114 have hoop ratios greater than the intermediate structural layer 113, and the innermost structural layer 112 has a hoop ratio greater than or less than the outermost structural layer 114. In one example (e.g., a PEBA tri-layer balloon), the innermost structural layer 112 includes PEBA, the intermediate structural layer 113 includes polyamide, copolyamide, or blends thereof, and the outer structural layer includes PEBA that is a PEBA-type different from the PEBA-type of the innermost structural layer. In another example (polyester TPE tri-layer balloon), the innermost and outermost structural layers 112, 114 each includes a polyester thermoplastic elastomer (polyester TPE), and the intermediate structural layer 113 includes a polyester or copolyester. Each of these examples is explained in more detail below.

In a particular example of the PEBA tri-layer balloon 110, the innermost and outermost structural layers 112, 114 each includes a PEBA, with the innermost and outermost structural layers 112, 114 have hoop ratios greater than the intermediate structural layer 113, and the innermost structural layer 112 has a hoop ratio greater than the outermost structural layer 114. As such, the PEBA of the innermost structural layer has a flexural modulus less than the respective flexural modulus of the PEBA included in the outermost structural layer, and the intermediate structural layer 113 includes a polyamide having a flexural modulus greater than the flexural modulus of each of the innermost and outermost structural layers 112, 114. Moreover in this same example, the PEBA of the innermost structural layer has a tensile strength at break less than the respective tensile strength at break of the PEBA included in the outermost structural layer, and the intermediate structural layer 113 includes a polyamide having a tensile strength at break less than the tensile strength at break of each of the innermost and outermost structural layers 112, 114. It is believed that this balloon body configuration allows for improved delivery through the body lumen, as compared to a balloon body of the same wall thickness having a single structural layer including the polyamide of the intermediate layer. Moreover, this configuration also facilitates wrapping and folding of the balloon.

Suitable material for the innermost structural layer 112 includes, but is not limited to, PEBAX 6333 SA 01 MED, PEBAX 6333 SA 01, PEBAX 7033 SA 01 MED, and PEBAX 7033 SA 01, each of which is commercially available from Arkema Inc. Properties of PEBAX 6333 SA 01 MED are provided below in Table 2 (below). Properties of PEBAX 7033 SA 01 MED are provided below in Table 3 (below). Suitable material for the intermediate structural layer 113 includes, but is not limited to, Grilamid® family of polyamides and copolyamides (e.g., Grilamid L25, Grilamid L20, Grilamid TR55 LX2, among others), commercially available from EMS-GRIVORY of Switzerland. Properties of Grilamid L25 are provided in Table 4 (below). Suitable material for the outermost structural layer 114 includes, but is not limited to, X-PEBA (disclosed above) and PEBAX 7233 SA 01 MED. Properties of PEBAX 7233 SA 01 MED are provided below in Table 5 (below).

TABLE 2

| Property | Typical Value | Unit | Test Method |
|---|---|---|---|
| Density | 1.01 | g/cm³ | ISO 1183 |
| Water Absorption at Equilibrium | | | |
| At 20° C. and 50% R.H. | .07 | % | ISO 62 |
| Water Absorption | | | |
| At 23° C. and 24 h in water | 1.1 | % | |
| Melting Point | 169 | ° C. | ISO 11357 |
| Vicat Point | | | |
| under 1 daN | 157 | ° C. | ISO 306 |
| Shrinkage (after 24 h, | | | |
| 4 mm, Mold at 40° C.) | | | |
| // | 1.2 | % | Internal |
| ⊥ | 1.4 | % | method |
| Hardness (*) | | | |
| Instantaneous | 64 | Shore D | ISO 868 |
| After 15 s | 58 | Shore D | |
| Tensile Test (*) | | | |
| Stress at Break | 53 | MPa | ISO 527 |
| Strain at Break | >350 | % | |
| Flexural Modulus (*) | 285 | MPa | ISO 178 |
| Charpy Impact (*) | | | |
| Unnotched 23° C. | No Break | kJ/M² | ISO 179 |
| Unnotched −30° C. | No Break | kJ/M2 | |
| V-Notched 23° C. | No Break | kJ/M² | |
| N-Notched −30° C. | 20 ^{(c)} | kJ/M² | |

TABLE 3

| Property | Typical Value | Unit | Test Method |
|---|---|---|---|
| Density | 1.01 | g/cm³ | ISO 1183 |
| Water Absorption at Equilibrium | | | |
| At 20° C. and 50% R.H. | .07 | % | ISO 62 |
| Water Absorption | | | |
| At 23° C. and 24 h in water | 1.1 | % | |
| Melting Point | 169 | ° C. | ISO 11357 |
| Vicat Point | | | |
| under 1 daN | 157 | ° C. | ISO 306 |
| Shrinkage (after 24 h, | | | |
| 4 mm, Mold at 40° C.) | | | |
| // | 1.2 | % | Internal |
| ⊥ | 1.4 | % | method |
| Hardness (*) | | | |
| Instantaneous | 64 | Shore D | ISO 868 |
| After 15 s | 58 | Shore D | |
| Tensile Test (*) | | | |
| Stress at Break | 53 | MPa | ISO 527 |
| Strain at Break | >350 | % | |
| Flexural Modulus (*) | 285 | MPa | ISO 178 |
| Charpy Impact (*) | | | |
| Unnotched 23° C. | No Break | kJ/M² | ISO 179 |
| Unnotched −30° C. | No Break | kJ/M2 | |
| V-Notched 23° C. | No Break | kJ/M² | |
| N-Notched −30° C. | 20 ^{(c)} | kJ/M² | |

TABLE 4
Product Texts
Product designation according to ISO 1874: PA 12, E, 24-010

| | dry/cond | Unit | Test Standard |
|---|---|---|---|
| Mechanical Properties | | | |
| Tensile Modulus | 1400/1100 | MPa | ISO 527-1/-2 |
| Yield stress | 45/40 | MPa | ISO 527-1/-2 |
| Yield Strain | 10/12 | % | ISO 527-1/-2 |
| Nominal strain at break | >50/>50 | % | ISO 527-1/-2 |
| Stress at break | 50/45 | MPa | ISO 527-1/-2 |
| Charpy impact strength | N/N | kJ/m² | ISO 179/1eU |
| (+23° C.) | | | |
| Charpy impact strength | N/N | kJ/m² | ISO 179/1eU |
| (−30° C.) | | | |
| Charpy notched impact | —/10 | kJ/m² | ISO 179/1eA |
| strength (+23° C.) | | | |
| Charpy notched impact | —/7 | kJ/m² | ISO 179/1eA |
| strength (−30° C.) | | | |
| Thermal Properties | | | |
| Melting temperature | 178/— | C | ISO 11357-1/-3 |
| (10° C./min) | | | |
| Other Properties | | | |
| Water absorption | 1.5/— | % | Sim. to ISO 62 |
| Humidity absorption | 0.7/— | % | Sim. to ISO 62 |
| Density | 1010/— | kg/m³ | ISO 1183 |
| Film Properties | | | |
| Stress at yield (parallel) | 35/— | MPa | ISO 527-3 |
| Stress at yield (normal) | 35/— | MPa | ISO 527-3 |
| Strain at yield (parallel) | 6/— | % | ISO 527-3 |
| Strain at yield (normal) | 6/— | % | ISO 527-3 |
| Maximum strain (parallel) | 850/— | % | ISO 527-3 |
| Maximum strain (normal) | 900/— | % | ISO 527-3 |
| Elmendorf Tear resistenace | 10/— | N | ISO 6383-2 |
| (parallel) | | | |
| Elmendorf Tear resistenace | 10/— | N | ISO 6383-2 |
| (normal) | | | |
| Trouser Tear resistance | 20/— | N/mm | ISO 6383-1 |
| (parallel) | | | |
| Trouser Tear resistance | 25/— | N/mm | ISO 6383-1 |
| (normal) | | | |
| Gloss, 60° | 150/— | — | ISO 2813 |
| WVTR (23° C./85% r.h.) | 8/— | g/(m²*d) | DIS 15106-1/-2 |
| Oxygen transmission rate | 350/— | cm^{3/}(m²*d*bar) | DIS 15105-1/-2 |
| (23° C./0% r.h.) | | | |
| Oxygen transmission rate | 370/— | cm^{3/}(m²*d*bar) | DIS 15105-1/-2 |
| (23° C./85% r.h.) | | | |
| Carbon Dioxide transm. | 1500/— | cm^{3/}(m²*d*bar) | DIS 15105-1/-2 |
| rate (23° C./0% r.h.) | | | |
| Carbon Dioxide transm. | 1600/— | cm^{3/}(m²*d*bar) | DIS 15105-1/-2 |
| rate (23° C./85% r.h.) | | | |
| Stress at break (parallel) | 80 | MPa | ISO 527-3 |
| Stress at break (normal) | 70 | MPa | ISO 527-3 |
| Gelbo flex test | 1300 | holes/m² | EMS |
| Rheo/Phys properties | | | |
| Melt volume-flow rate | 20/— | cm³/10 min | ISO 1133 |
| (MVR) | | | |
| Temperature | 275/— | ° C. | ISO 1133 |
| Load | 5/— | kg | ISO 1133 |

TABLE 5

| Property | Typical Value | Unit | Test Method |
|---|---|---|---|
| Density | 1.01 | g/cm³ | ISO 1183 |
| Water Absorption at Equilibrium | | | |
| At 20° C. and 50% R.H. | .07 | % | ISO 62 |
| Water Absorption | | | |
| At 23° C. and 24 h in water | 0.9 | % | |
| Melting Point | 174 | ° C. | ISO 11357 |
| Vicat Point | | | |
| under 1 daN | 164 | ° C. | ISO 306 |
| Shrinkage (after 24 h, | | | |
| 4 mm, Mold at 40° C.) | | | |
| // | 1.2 | % | Internal |
| ⊥ | 1.5 | % | method |
| Hardness (*) | | | |
| Instantaneous | 69 | Shore D | ISO 868 |
| After 15 s | 61 | Shore D | |
| Tensile Test (*) | | | |
| Stress at Break | 56 | MPa | ISO 527 |
| Strain at Break | >300 | % | |
| Flexural Modulus (*) | 513 | MPa | ISO 178 |
| Charpy Impact (*) | | | |
| Unnotched 23° C. | No Break | kJ/M² | ISO 179 |
| Unnotched −30° C. | No Break | kJ/M2 | |
| V-Notched 23° C. | 15 ^{(P)} | kJ/M² | |
| N-Notched −30° C. | 10 ^{(c)} | kJ/M² | |

In one example of the tri-layer PEBA balloon 110, the body 116 of the balloon may have a maximum outer diameter OD2 (upon inflation or expansion) from about 3.0 mm to about 12.0 mm. For example, the balloon body diameter OD2 may be about 3.0 mm, or about 3.5 mm, or about 4.0 mm, or about 4.5 mm, or about 5.0 mm, or about 5.5 mm, or about 6.0 mm, or about 6.5 mm, or about 7.0 mm, or about 7.5 mm, or about 8.0 mm, or about 8.5 mm, or about 9.0 mm, or about 9.5 mm, or about 10.0 mm, or about 10.5 mm, or about 11.0 mm, or about 11.5 mm, or about 12.0 mm, or about 12.5 mm, among other diameters. The body 116 of the tri-layer PEBA balloon 110 may have a working length WL2 (FIG. 3) from about 15 mm to about 200 mm. The body 116 of the tri-layer PEBA balloon 110 has a total wall thickness T2 (FIG. 4) of about 0.025 inch (0.635 mm). In other examples, the total wall thickness T2 of the body 116 may be from about 0.015 inch (0.381 mm) to about 0.040 inch (1.02 mm), or from about 0.020 inch (0.508 mm) to about 0.030 inch (0.762 mm), or from about 0.020 inch (0.508 mm) to about 0.025 inch (0.635 mm). A thickness of the outer structural layer 114 may be about 25% of the total wall thickness T2 of the body 116, a thickness of the inner structural layer 112 may be about 50% of the total wall thickness of the body, and a thickness of the intermediate structural layer 113 may be about 25% of the total wall thickness of the body. In other examples, the respective percent thicknesses of the inner structural layer 112/the intermediate structural layer 113/the outer structural layer 114 relative to the total thickness T2 may be one of the following (as percentages): 25/50/25; 15/70/15; 40/20/40.

In one exemplary method, the tri-layer layer PEBA balloon 110 may be blow-molded from a preform or parison using blow molding techniques and processes or by other methods. The parison may be formed by co-extruding the inner, intermediate, and outer structural layers 112, 113, 114 using coextrusion techniques and processes or by other methods. In at least one example, the body 116 is free from a tie or adhesive layer between the three structural layers 112, 113, 114.

Referring still to FIGS. 3 and 4, in another example the innermost and outermost structural layers 112, 114 each includes a polyester thermoplastic elastomer (polyester TPE), and the intermediate structural layer 113 includes a polyester or copolyester. With respect to the innermost and outermost structural layers 112, 114 of this tri-layer polyester TPE balloon example, the polyester TPE may be a block copolymer comprising a hard segment of polyester (e.g., polybutylene terephthalate (PBT)) and a soft segment of polyether. A suitable polyester TPE is HYTREL®, which is commercially available from E. I. du Pont de Nemours and Company, Wilmington, Del. The polyester TPE may have a Shore D hardness from 30D to 82D. In one example, the polyester TPE of the innermost structural layer 112 has a lower Shore D hardness than the polyester TPE of the outermost structural layer 114. One or both of the innermost and outermost structural layers 112, 114 may consist essentially of the polyester block copolymer (e.g., HYTREL) or a blend thereof, such as set forth above with respect to the corresponding polyester TPE balloon of FIGS. 1 and 2.

Suitable material for the intermediate structural layer 113 includes, but is not limited to, ARRAY 9921M copolyester, commercially available from DAK Americas. The outer structural layer 14 may consist essentially of the copolyester (e.g., ARRAY 9921M copolyester) or a blend thereof. The outer structural layer 14 may include other types of copolyesters or other types of polymers.

In one example of the tri-layer polyester TPE balloon 110, the body 116 of the balloon may have a maximum outer diameter OD2 (upon inflation or expansion) from about 3.0 mm to about 12.0 mm. For example, the balloon body 116 OD2 may be about 3.0 mm, or about 3.5 mm, or about 4.0 mm, or about 4.5 mm, or about 5.0 mm, or about 5.5 mm, or about 6.0 mm, or about 6.5 mm, or about 7.0 mm, or about 7.5 mm, or about 8.0 mm, or about 8.5 mm, or about 9.0 mm, or about 9.5 mm, or about 10.0 mm, or about 10.5 mm, or about 11.0 mm, or about 11.5 mm, or about 12.0 mm, or about 12.5 mm, among other diameters. The body 116 of the tri-layer polyester TPE balloon 110 may have a working length WL2 (FIG. 3) from about 15 mm to about 200 mm. The body 116 of the tri-layer polyester TPE balloon 110 has a total wall thickness T2 (FIG. 4) of about 0.020 inch (0.635 mm). In other examples, the total wall thickness T2 of the body 116 may be from about 0.015 inch (0.381 mm) to about 0.040 inch (1.02 mm), or from about 0.020 inch (0.508 mm) to about 0.030 inch (0.762 mm), or from about 0.020 inch (0.508 mm) to about 0.025 inch (0.635 mm). A thickness of the outer structural layer 114 may be about 25% of the total wall thickness T2 of the body 116, a thickness of the inner structural layer 112 may be about 50% of the total wall thickness of the body, and a thickness of the intermediate structural layer 113 may be about 25% of the total wall thickness of the body. In other examples, the respective percent thicknesses of the inner structural layer 112/the intermediate structural layer 113/the outer structural layer 114 relative to the total thickness T2 may be one of the following (as percentages): 25/50/25; 15/70/15; 40/20/40.

In one exemplary method, the tri-layer layer polyester TPE balloon 110 may be blow-molded from a preform or parison using conventional blow molding techniques and processes or by other methods. The parison may be formed by co-extruding the inner, intermediate, and outer structural layers 112, 113, 114 using conventional coextrusion techniques and processes or by other methods. In at least one example, the body 116 is free from a tie or adhesive layer between the three structural layers 112, 113, 114.

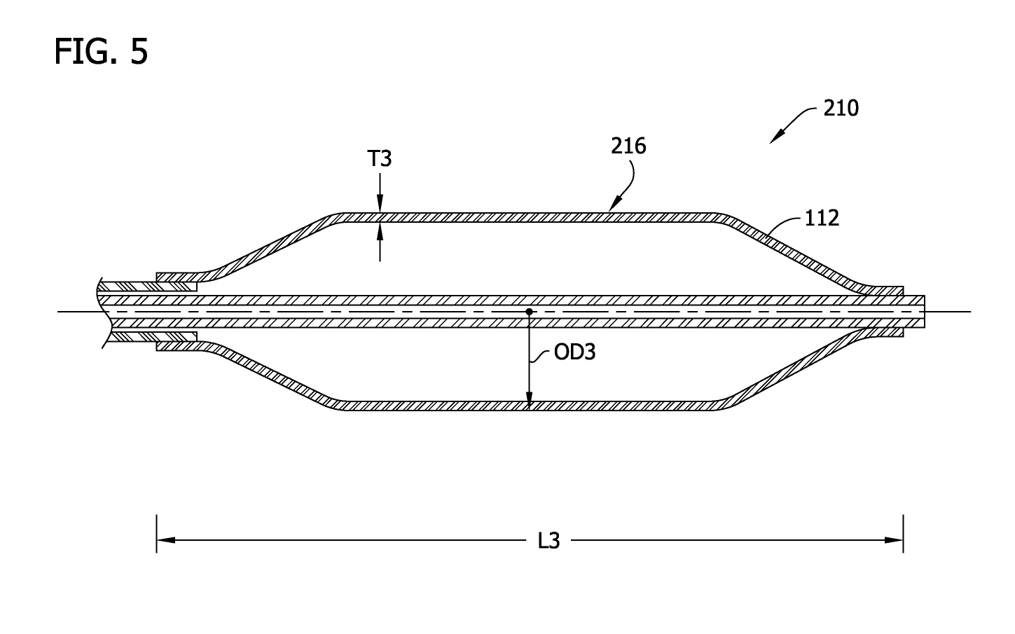

Referring to FIG. 5, in another non-limiting embodiment of the present disclosure, a body 216 of a transluminal balloon 210 for a medical device comprising a single structural layer 112, i.e., one and only one structural layer (hereinafter referred to as a “monolayer balloon”).

As set forth above, in one example the single structural layer of the monolayer balloon may include X-PEBA. For example, the single structural layer may consist essentially of X-PEBA or a blend thereof. With respect to the X-PEBA blend, X-PEBA may be blended with polyamide. Exemplary polyamides for use in the X-PEBA blend include, but are not limited to, aliphatic polyamides, such as nylon 12; nylon 11; nylon 9; nylon 6; nylon 6, 12; nylon 6, 11; nylon 6, 9; nylon 6,6; nylon 10, 10; nylon 10, 12; nylon 6, 10; nylon 13; nylon 6, 13; nylon 10, 13; nylon 11; nylon 6, 11; or nylon 10, 11; or aromatic polyamides. Additional exemplary polyamides include transparent amorphous polyamides having a segment such as an aliphatic segment, an aromatic segment, or a cycloaliphatic segment. The selected transparent amorphous polyamide may have a Shore D durometer value greater than 80, and a glass transition temperature greater than 100° C. or greater than 120° C. A suitable, non-limiting example of transparent amorphous polyamide includes GRILAMID® TR55 LX2, commercially available from EMS-GRIVORY of Switzerland. The transparent amorphous polyamide may have the following properties provided in Table 1. The X-PEBA blend may include other types of polyamides and/or may include other types of polymers. In one example, the X-PEBA blend may be more than 50% by weight X-PEBA, or more than or equal to about 60% by weight X-PEBA, or more than or equal to about 70% by weight X-PEBA, or more than or equal to about 80% by weight X-PEBA, or more than or equal to about 85% by weight X-PEBA, or more than or equal to 90% by weight X-PEBA, or more than or equal to 95% by weight X-PEBA.

The body 216 of the balloon may have a maximum outer diameter OD3 (upon inflation or expansion) from about 3.0 mm to about 12.0 mm. For example, the balloon body outer diameter OD3 may be about 3.0 mm, or about 3.5 mm, or about 4.0 mm, or about 4.5 mm, or about 5.0 mm, or about 5.5 mm, or about 6.0 mm, or about 6.5 mm, or about 7.0 mm, or about 7.5 mm, or about 8.0 mm, or about 8.5 mm, or about 9.0 mm, or about 9.5 mm, or about 10.0 mm, or about 10.5 mm, or about 11.0 mm, or about 11.5 mm, or about 12.0 mm, or about 12.5 mm, among other diameters. The body 216 of the monolayer X-PEBAX balloon 210 may have a working length WL3 (FIG. 5) from about 15 mm to about 200 mm. The body 216 has a total wall thickness T3 (FIG. 6) of about 0.025 inch (0.635 mm). In other examples, the total wall thickness T3 of the body 216 may be from about 0.015 inch (0.381 mm) to about 0.040 inch (1.02 mm), or from about 0.020 inch (0.508 mm) to about 0.030 inch (0.762 mm), or from about 0.020 inch (0.508 mm) to about 0.025 inch (0.635 mm).

In another example of the monolayer balloon 210, the single structural layer may consist essentially of transparent amorphous polyamide or a blend thereof. With respect to the transparent amorphous polyamide blend, transparent amorphous polyamide may be blended with polyamide. Exemplary polyamides for use in the transparent amorphous polyamide blend include, but are not limited to, aliphatic polyamides, such as nylons 12, 6, 12; and 10, 12; or aromatic polyamides. A suitable, non-limiting example of transparent amorphous polyamide includes GRILAMID® TR55 LX2, commercially available from EMS-GRIVORY of Switzerland. The transparent amorphous polyamide may have the following properties provided in Table 1. The transparent amorphous polyamide blend may include other types of polyamides and/or may include other types of polymers. In one example, the transparent amorphous polyamide blend may be more than 50% by weight transparent amorphous polyamide, or more than or equal to about 60% by weight transparent amorphous polyamide, or more than or equal to about 70% by weight transparent amorphous polyamide, or more than or equal to about 80% by weight transparent amorphous polyamide, or more than or equal to about 85% by weight transparent amorphous polyamide, or more than or equal to 90% by weight transparent amorphous polyamide, or more than or equal to 95% by weight transparent amorphous polyamide.

The body 216 of the balloon may have a maximum outer diameter OD3 (upon inflation or expansion) from about 3.0 mm to about 12.0 mm. For example, the balloon body outer diameter OD3 may be about 3.0 mm, or about 3.5 mm, or about 4.0 mm, or about 4.5 mm, or about 5.0 mm, or about 5.5 mm, or about 6.0 mm, or about 6.5 mm, or about 7.0 mm, or about 7.5 mm, or about 8.0 mm, or about 8.5 mm, or about 9.0 mm, or about 9.5 mm, or about 10.0 mm, or about 10.5 mm, or about 11.0 mm, or about 11.5 mm, or about 12.0 mm, or about 12.5 mm, among other diameters. The body 216 of the monolayer transparent amorphous polyamide balloon 210 may have a length WL3 (FIG. 5) from about 15 mm to about 200 mm. The body 216 has a total wall thickness T3 (FIG. 5) of about 0.020 inch (0.635 mm). In other examples, the total wall thickness T3 of the body 216 may be from about 0.015 inch (0.381 mm) to about 0.040 inch (1.02 mm), or from about 0.020 inch (0.508 mm) to about 0.030 inch (0.762 mm), or from about 0.020 inch (0.508 mm) to about 0.025 inch (0.635 mm).

Throughout the above disclosure, in other examples of the monolayer and multilayer balloons, nylon 12 in one or more of the structural layers is replaced with transparent amorphous polyamide, such as GRILAMID® TR55 LX2, disclosed above.

Having described the invention in detail, it will be apparent that modifications and variations are possible without departing from the scope of the invention defined in the appended claims.

When introducing elements of the present invention or the preferred embodiment(s) thereof, the articles “a”, “an”, “the” and “said” are intended to mean that there are one or more of the elements. The terms “comprising,” “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A transluminal balloon for a medical device comprising:
a longitudinal structural portion comprising at least a first structural layer, the first structural layer comprising a polyether block amide co-polymer comprising a polyether, and a polyamide other than nylon 12.

2. The transluminal balloon set forth in claim 1, wherein the polyamide of the polyether block amide co-polymer of the first structural layer is formed from oligomerization of a monomer other than laurolactam.

3. The transluminal balloon set forth in claim 1, wherein the polyamide of the polyether block amide co-polymer of the first structural layer comprises one or more of nylon 11; nylon 13; nylon 6, 11; nylon 6, 13; nylon 10, 11; and nylon 10, 13.

4. The transluminal balloon set forth in claim 1, wherein the Shore D hardness of the polyether block amide co-polymer of the first structural layer is from about 72D to about 75D.

5. The transluminal balloon set forth in claim 2, wherein the Shore D hardness of the polyether block amide co-polymer of the first structural layer is about 74D.

6. The transluminal balloon set forth in claim 1, wherein the longitudinal structural portion comprises a second structural layer generally coextensive with the first structurally layer, the second structural layer comprising a polyamide.

7. The transluminal balloon set forth in claim 6, wherein the first structural layer is an inner structural layer and the second structural layer is an outer structural layer.

8. The transluminal balloon set forth in claim 7, wherein the longitudinal structural portion is free from other structural layers such that the transluminal balloon is a bi-layer balloon.

9. The transluminal balloon set forth in claim 1, wherein the longitudinal structural portion is free from other structural layers such that the transluminal balloon is a monolayer balloon.

10. The transluminal balloon set forth in claim 1, wherein the first structural layer further comprises a polyamide blended with the polyether block amide co-polymer.

11. The transluminal balloon set forth in claim 10, wherein the polyamide of the first structural layer comprises at least one of an aliphatic polyamide, or an aromatic polyamide, or a transparent amorphous polyamide.

12. The transluminal balloon set forth in claim 10, wherein the polyamide of the first structural layer comprises a transparent amorphous polyamide having a Shore D durometer value greater than 80D and a glass transition temperature greater than 100° C.

13. A transluminal balloon for a medical device comprising:
a longitudinal structural portion comprising:
an inner structural layer comprising a first polyether block amide co-polymer;
an intermediate structural layer comprising a polyamide or copolyamide;
an outer structural layer a second polyether block amide co-polymer different from the first polyether block amide co-polymer;
wherein the inner and outer structural layers have hoop ratios greater than the intermediate structural layer, and the inner structural layer has a hoop ratio that is greater than or less than the outer structural layer.

14. The transluminal balloon set forth in claim 13, wherein the inner structural layer consists essentially of the first polyether block amide co-polymer, the outer structural layer consists essentially of the second polyether block amide co-polymer, and the intermediate layer consists essentially of the polyamide or copolyamide.

15. The transluminal balloon set forth in claim 13, wherein the first polyether block amide co-polymer has a flexural modulus less than a flexural modulus of the second polyether block amide co-polymer, and the polyamide or copolyamide of the intermediate structural layer has a flexural modulus greater than the flexural modulus of each of the first and second polyether block amide co-polymers.

16. The transluminal balloon set forth in claim 13, wherein the first polyether block amide co-polymer has a tensile strength at break less than a tensile strength at break of the second polyether block amide co-polymer, and the polyamide or copolyamide of the intermediate structural layer has a tensile strength at break less than the tensile strength at break of each of the first and second polyether block amide co-polymers.

17. The transluminal balloon set forth in claim 13, wherein the longitudinal structural portion consists essentially of the inner, outer and intermediate structural layers.

18. A transluminal balloon for a medical device comprising
a longitudinal structural portion comprising:
an inner structural layer comprising a first polyester block copolymer thermoplastic elastomer comprising a hard segment of polyester and a soft segment of polyether;
an intermediate structural layer comprising a polyester or copolyester;
an outer structural layer comprising a second polyester thermoplastic block copolymer comprising a hard segment of polyester and a soft segment of polyether,
wherein the first polyester block copolymer thermoplastic elastomer has a lower Shore D hardness than a Shore D hardness of the second polyester block copolymer thermoplastic elastomer polyester.

19. The transluminal balloon set forth in claim 18, wherein the hard segments of polyester of the inner and outer structural layers comprise polybutylene terephthalate.

20. The transluminal balloon set forth in claim 18, wherein each of the inner and outer structural layers consists essentially of the corresponding one of the first and second polyester block copolymers.
